(19) United States
(12) Patent Application Publication
(10) Pub. No.: US 2026/0263601 A1
(43) Pub. Date: Sep. 10, 2026

(54) DUAL-COMPOSITE DELIVERY SYSTEM

(71) Applicant: University of Washington, Seattle, WA (US)

(72) Inventors: Duru Tasman, Seattle, WA (US); Kim Woodrow, Seattle, WA (US); Shin-Tian Chien, Seattle, WA (US); Hannah VanBenschoten, Seattle, WA (US); Ian Suydam, Seattle, WA (US)

(73) Assignee: University of Washington, Seattle, WA (US)

(21) Appl. No.: 19/542,553

(22) Filed: Feb. 17, 2026

Related U.S. Application Data
(60) Provisional application No. 63/761,650, filed on Feb. 21, 2025.

(51) Int. Cl. A61K 47/34; A61K 31/475; A61K 31/513

(52) U.S. Cl. CPC A61K 47/34; A61K 31/475; A61K 31/513

(57) ABSTRACT

A drug delivery system, including a swelling polymer matrix, and a polymerized hydrophilic drug system is configured to control the release of the hydrophilic drug. Additionally, a method of using the drug delivery system 1, the method including providing the polymerized hydrophilic drug dispersed within the swelling polymer matrix, reaching a water equilibrium within the swelling polymer matrix, hydrolyzing the polymerized hydrophilic drug, and diffusing the hydrophilic drug outside of the swelling polymer matrix Further, A method of making the drug delivery system, the method including polymerizing a hydrophilic drug to produce a polymerized hydrophilic drug, and embedding the polymerized hydrophilic drug into a swelling polymer matrix.

### CROSS-REFERENCE TO RELATED APPLICATION

This application claims the benefit of U.S. Provisional Application No. 63/761,650, filed Feb. 21, 2025, the entire disclosures of which are hereby incorporated by reference.

### STATEMENT OF GOVERNMENT LICENSE RIGHTS

This invention was made with government support under Grant No. R01AI145483, awarded by the National Institutes of Health. The government has certain rights in the invention.

### BACKGROUND

The gold standard for long-acting formulations is zero-order release, providing the necessary daily therapeutic dose without local or systemic toxicity. Zero-order release also prevents quick clearance of large amounts of drugs.

Hydrophilic drugs are categorized as having a narrow therapeutic window due to their quick clearance from the body as they are more easily metabolized by the body. This can make it difficult for hydrophilic drugs to achieve zero-order release. This is complicated even further when hydrophilic drugs are used in swelling systems. Swelling systems are when the whole delivery system allows water diffusion throughout, where the drug molecules are embedded throughout.

It is desirable to deliver hydrophilic drugs with swelling systems, but their narrow therapeutic window often limits their use in such systems as they may release much quicker than desired. Within diffusion-mediated delivery systems, water-soluble drugs often result in a biphasic release, where a burst effect is followed by sustained delivery depending on the carrier hydrophilicity.

Accordingly, drug delivery systems and methods for making swelling systems embedded with hydrophilic drugs that release of the water-soluble drugs in a controlled manner to maintain a necessary concentration of the hydrophilic drug within the blood are needed.

### SUMMARY

In one aspect, disclosed herein is a drug delivery system, including a swelling polymer matrix, and a polymerized hydrophilic drug, wherein the drug delivery system is configured to control the release of the hydrophilic drug.

In some embodiments, the drug delivery system is configured to achieve a near zero-order release of the hydrophilic drug. In some embodiments, the drug delivery system is configured to achieve a zero-order release of the hydrophilic drug. In some embodiments, the drug delivery system is hydrophilic drug has an R2 value greater than 0.949.

In some embodiments, the hydrophilic drug is polymerized with reversible addition fragmentation chain transfer (RAFT) polymerization.

In some embodiments, controlling the release of the hydrophilic drug includes reaching a water equilibrium within the swelling polymer matrix, hydrolyzing the polymerized hydrophilic drug within the swelling polymer matrix, and diffusing the hydrophilic drug from the swelling polymer matrix.

In some embodiments, the hydrophilic drug has a molecular weight of about 100-1000 g/mol (0.1-1 kDa). In some embodiments, the hydrophilic drug is raltegravir (RAL). In some embodiments, the hydrophilic drug is 10-hydroxycampthotecin (CMP). In some embodiments, the polymerized hydrophilic drug comprises comprising a polymeric backbone with hydrolytic linkers. In some embodiments, the hydrolytic linkers are ester linkers. In some embodiments, the hydrolytic linkers are acetal carbonate linkers.

In some embodiments, the swelling polymer matrix is a Poly(lactic-co-glycolic acid) (PLGA) matrix. In some embodiments, the swelling polymer matrix is a thermoplastic polyurethane (TPU) matrix. In some embodiments, the TPU matrix is a high-swelling (HS) TPU matrix. In some embodiments, the TPU matrix is selected from PT42DE35 and PY-PT83AE35.

In some embodiments, the drug delivery system is configured to release the hydrophilic drug over a duration of about 30 days.

In another aspect, disclosed herein is a method of using the drug delivery system disclosed herein, the method including providing the polymerized hydrophilic drug dispersed within the swelling polymer matrix, reaching a water equilibrium within the swelling polymer matrix, hydrolyzing the polymerized hydrophilic drug, and diffusing the hydrophilic drug outside of the swelling polymer matrix. In some embodiments, the hydrophilic drug is released at an effective level for about 30 days.

In some embodiments, the method includes polymerizing a hydrophilic drug to produce a polymerized hydrophilic drug, and embedding the polymerized hydrophilic drug into a swelling polymer matrix.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIGS. 1A-1B are an example drug delivery system and its theoretical drug release over time in accordance with the present technology;

FIG. 2 is an example drug delivery system including a raltegravir (RAL) molecule RAFT polymerized to a polymer backbone with a hydrolytic linker in accordance with the present technology;

FIG. 3 is a P(RALSMA)13k hydrolysis scheme with their respective molecular weights (MWs) in accordance with the present technology;

FIGS. 4A-4B are graphs showing cumulative and daily RAL release from RAL and p(RALSMA)13k embedded in HS-TPU separately in accordance with the present technology;

FIGS. 5A-5H show an example release mechanism with its phases, for an example dual-composite delivery system in accordance with the present technology;

FIG. 6 shows swelling data over time for blank TPU devices in accordance with the present technology;

FIG. 7 is the cumulative RAL-ISO release from RAL-ISO embedded in HS-TPU in PBS media in accordance with the present technology;

FIGS. 8A-8C show example drug delivery systems including p(RALSMA)13k and RAL in HS-TPU, respectively in accordance with the present technology.

FIG. 9 is a graph showing RAL concentrations in PBS (pH=7.4) throughout the release study for RAL and p(RALSMA)13k in MS-TPU, HS-TPU, and LS-TPU at 37° C. in accordance with the present technology.

FIG. 10 is a graph showing the cumulative release of RAL and p(RALSMA)13k over time in days in accordance with the present technology.

FIGS. 11A-11B are graphs of the location of p(RALSMA)13k, p(RALSMA)4.5k, and RAL throughout the release study in accordance with the present technology;

FIG. 12 is a graph showing the location of RAL each day in accordance with the present technology;

FIG. 13 is a graph showing the cumulative RAL release over time in multiple polymer matrices, in accordance with the present technology;

FIG. 14 is a graph showing the cumulative backbone release over time in multiple polymer matrices, in accordance with the present technology;

FIG. 15 is a graph showing the cumulative RAL release over time in multiple polymer matrices, in accordance with the present technology;

FIG. 16 is a graph showing drug release per day, in accordance with the present technology;

FIG. 17 is a graph showing RAL release over time, in accordance with the present technology;

FIG. 18 is a graph showing RAL release over time, in accordance with the present technology;

FIG. 19 is a graph showing the cumulative backbone release over time in multiple polymer matrices, in accordance with the present technology;

FIG. 20 is a graph showing RAL release over time, in accordance with the present technology;

FIG. 21 is a graph showing RAL release over time, in accordance with the present technology;

FIG. 22 is a graph showing RAL release over time, in accordance with the present technology;

FIG. 23A-23B are graphs showing cumulative RAL release over time, in accordance with the present technology;

FIG. 24A-24B are graphs showing cumulative CMP release over time, in accordance with the present technology; and

FIG. 25A-25B are graphs showing cumulative RAL release and cumulative backbone release over time, in accordance with the present technology.

### DETAILED DESCRIPTION

Disclosed herein is a drug delivery system, including a swelling polymer matrix, and a polymerized hydrophilic drug, where the drug delivery system is configured to achieve a zero-order release of the hydrophilic drug.

In one embodiment, disclosed herein is a dual-composite drug delivery system (also referred to herein as a “drug delivery system”) including a hydrophilic (or water-soluble) drug that is reversible addition fragmentation chain transfer (RAFT) polymerized and then embedded in a thermoplastic polyurethane (TPU) matrix to control the water-soluble drug's release profile.

RAFT polymerization may be used to create a drug-polymer conjugate (having a polymer backbone and linkers) due to its control over polymeric dispersity but also its versatility with chemical conditions, radical initiators, and chain transfer agents, which allow for hydrophilic drug conjugation. RAFT polymers have been shown to create in-situ depot delivery systems. However, in-situ depots present logistical variables that can impact the release significantly, such as the injection volume and how fast it was injected. Additionally, molecular weight (MW) and degree of polymerization (DP) are also relevant for depot formations. With the systems disclosed herein, once a certain MW threshold is reached, the dispersity may not matter much.

The dual-composite delivery systems disclosed herein differ from other diffusion-mediated systems by including RAFT polymerization, which introduces a hydrolysis reaction for drug release, achieving near zero-order release.

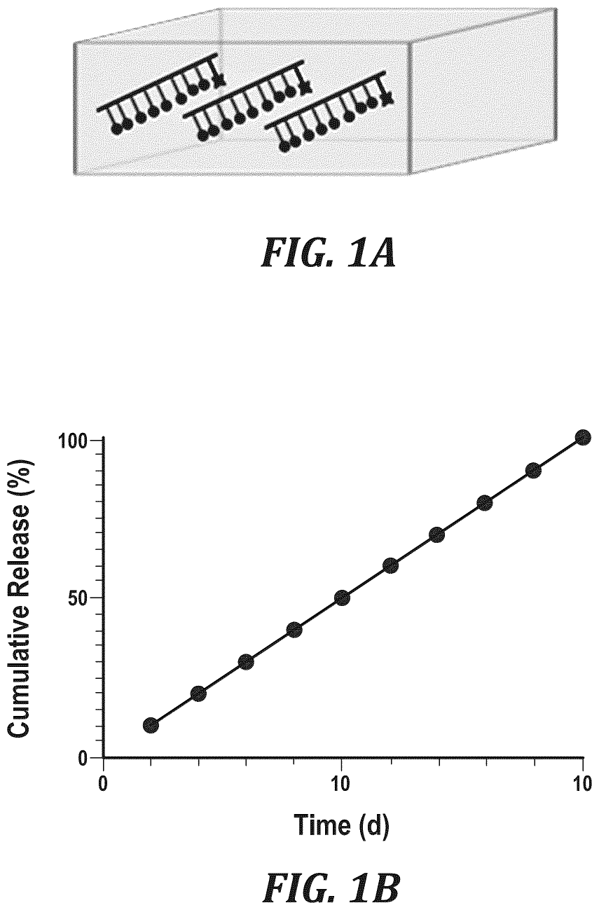

FIGS. 1A-1B are (A) an example drug delivery system and (B) its theoretical release over time. As shown, the example drug delivery system of FIG. 1A includes a hydrophilic drug (red circles), RAFT polymerized to a polymer backbone (black) with hydrolytic linkers (green).

In one aspect, disclosed herein is a drug delivery system, including a thermoplastic polyurethane (TPU) matrix, and a reversible addition fragmentation chain transfer (RAFT) polymerized hydrophilic drug comprising a polymeric backbone with hydrolytic linkers, where the drug delivery system is configured to control the release of the hydrophilic drug.

In the drug delivery system is configured to achieve a near zero-order release of the hydrophilic drug.

In some the drug delivery system is configured to achieve a zero-order release of the hydrophilic drug.

In some embodiments, achieving the zero-order release of the hydrophilic drug includes reaching a water equilibrium within the TPU matrix, hydrolyzing the RAFT polymerized hydrophilic drug, and diffusing the hydrophilic drug from the TPU matrix.

In some embodiments, the hydrophilic drug has a molecular weight of about 400 to 500 g/mol. In some embodiments, the hydrophilic drug is raltegravir (RAL). In some embodiments, the hydrolytic linkers are ester linkers.

In some embodiments, the TPU matrix is a high-swelling (HS) TPU matrix. In some embodiments, the HS-TPU matrix is selected from PT42DE35 and PY-PT83AE35.

In some embodiments, the drug delivery system is configured to release the hydrophilic drug at an effective level over a duration of about 30 days.

In another aspect, disclosed herein is a method of using the drug delivery system of disclosed herein, including providing the RAFT polymerized hydrophilic drug dispersed within the TPU matrix, reaching a water equilibrium within the TPU matrix, hydrolyzing the RAFT polymerized hydrophilic drug, and diffusing the hydrophilic drug outside of the TPU matrix.

In some embodiments, hydrolyzing the RAFT polymerized hydrophilic drug comprises cleaving the hydrophilic drug from the polymer backbone and hydrolytic linkers.

In some embodiments, the hydrophilic drug has a molecular weight of about 400 to 500 g/mol. In some embodiments, the hydrophilic drug is raltegravir (RAL). In some embodiments, the hydrophilic drug is 10-hydroxycampthotecin (CMP).

In some embodiments, the hydrolytic linkers are ester linkers. In some embodiments, the hydrolytic linkers are acetal carbonate linkers.

In some embodiments, the TPU matrix is a high-swelling (HS-)TPU matrix. In some embodiments, the hydrophilic drug is released at an effective level for about 30 days.

In yet another aspect, disclosed herein is a method making a drug delivery system, including reversible addition fragmentation chain transfer (RAFT) polymerizing a hydrophilic drug to produce a RAFT polymerized hydrophilic drug comprising a polymer backbone with hydrolytic linkers, and embedding the RAFT polymerized hydrophilic drug into a thermoplastic polyurethane (TPU) matrix.

In some embodiments, the hydrophilic drug has a molecular weight of about 400 to 500 g/mol. In some embodiments, the hydrophilic drug is raltegravir (RAL). In some embodiments, the hydrolytic linkers are ester linkers.

In some embodiments, the TPU matrix is a high-swelling(HS-)TPU matrix.

### Examples

In one example, the hydrophilic drug (also referred to herein as an “active pharmaceutical ingredient” or “API”) is RAL (HIV integrase inhibitor). RAL was selected due to its prevalence in combinational therapies for HIV prevention and treatment, and also its modifications to increase its retention within the body, since it is an easily metabolized small water-soluble drug.

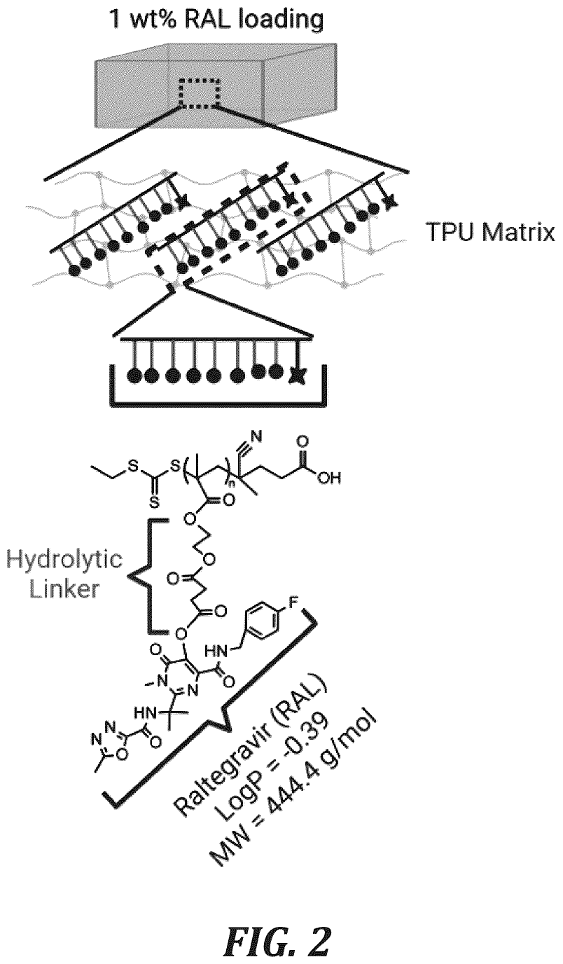

FIG. 2 is an example drug delivery system including a raltegravir (RAL) molecule RAFT polymerized to a polymer backbone with a hydrolytic linker. Also shown is the molecular weight (MW) and the Log P value of RAL.

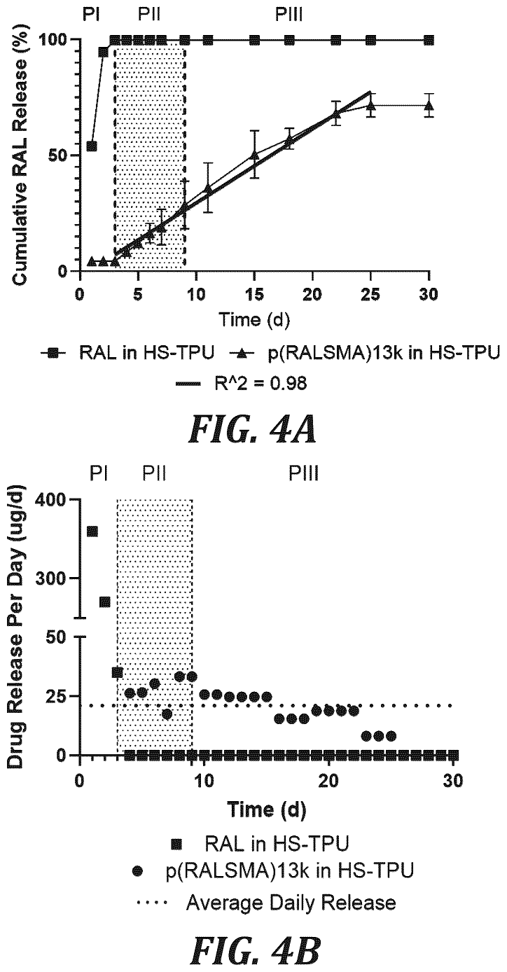

When initially observed, RAL embedded in HS-TPU resulted in a burst effect, with 50% release happening within the first day (FIG. 4A), and no release observed after the third day (FIG. 4B).

A dual-composite delivery system configured to provide various levers to manipulate the drug dissolution according to individual needs via changing TPU, polymer, and linker chemistry was developed. As detailed herein, RAFT polymerization with an ester linker may prevent any burst effect from the TPU matrix due to the introduced hydrolysis requirement. Further, TPU matrix hydrophilicity may further control the drug release profile while maintaining the diffusion-mediated release mechanics.

RAFT polymerization of RAL, p(RALSMA)13k, by linking the drug molecules to each other via a polymeric backbone with ester linkers, introduced the hydrolysis reaction for RAL release within the diffusion-meditated TPU system. The linker cleavage prerequisite and notable MW increase were among the reasons for the release profile change with p(RALSMA)13k in HS-TPU.

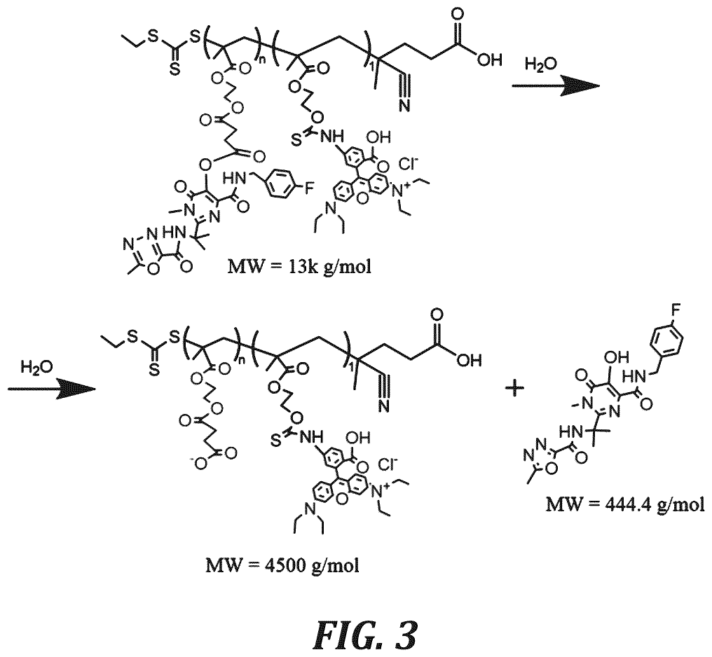

FIG. 3 is a P(RALSMA)13k hydrolysis scheme with their respective molecular weights (MWs). The RAL-polymer and cleaved backbone MWs are approximations from calculations. Red=RAL, Green=SMA, Pink=RhMA, and Black=polymeric backbone.

FIGS. 4A-4B are graphs showing cumulative and daily RAL release from RAL and p(RALSMA)13k embedded in high swelling-(HS-)TPU separately. (A) RAL concentrations in PBS (pH=7.4) throughout the release study for RAL and p(RALSMA)13k in HS-TPU. (B) Daily RAL release per day calculated by dividing sample averages according to the collected timepoint periods for RAL and p(RALSMA)13k in HS-TPU.

In contrast to RAL embedded in HS-TPU, the dual-composite system achieved near-zero order release with 50% release happening by day 15 (FIG. 4A) and having continuous daily average releases between the third and 25th days of the month-long investigation (FIG. 4B). It was observed that full saturation was needed for hydrolysis with the dual-composite system, as there was a lag period of three days before any RAL release (FIG. 4A).

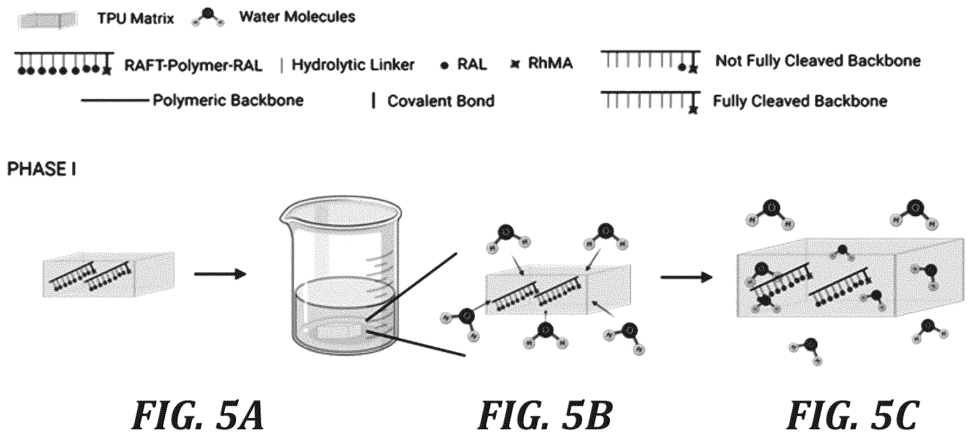

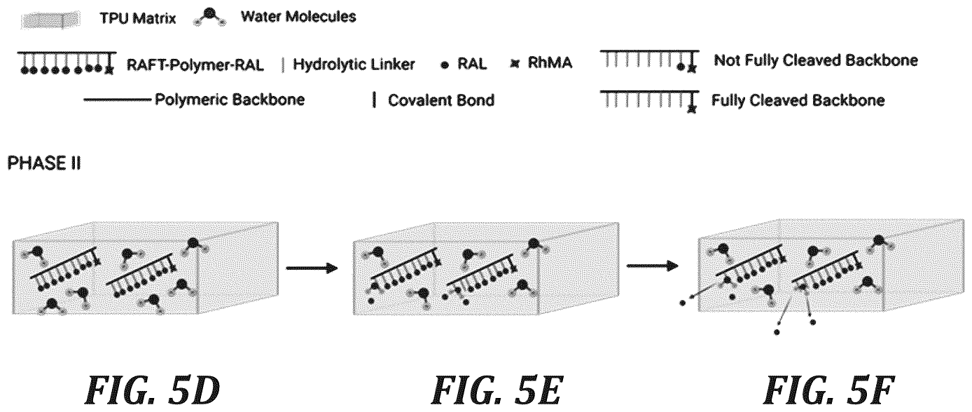

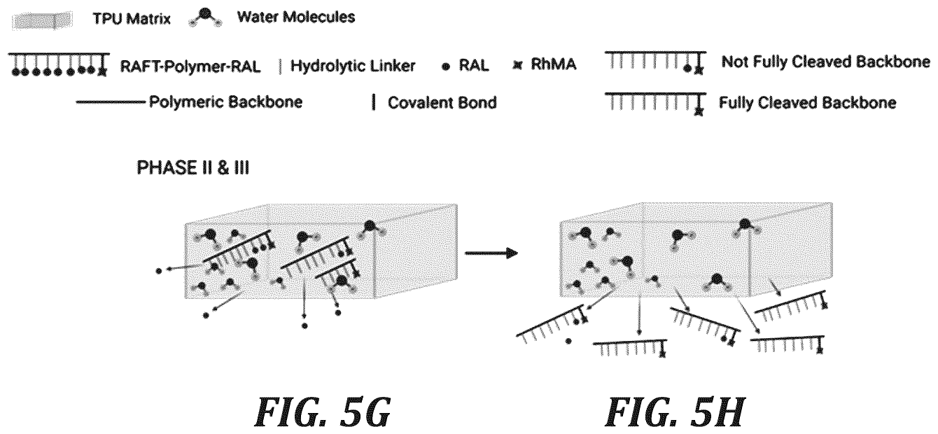

The lag period coincided with the time needed to reach maximum swelling for HS-TPU, which is depicted in Phase I of Scheme I (FIGS. 5A-5H).

FIG. 5A-5H show an example release mechanism with its phases, for an example dual-composite delivery system. PHASE I=Water Equilibrium, PHASE II=Hydrolysis and RAL release, and PHASE III=RAL and backbone release.

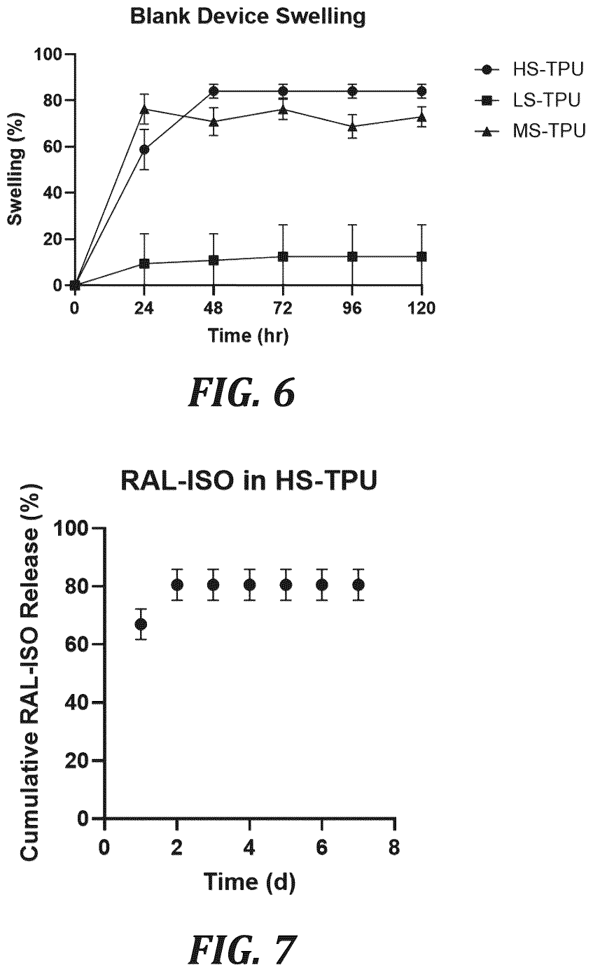

FIG. 6 shows swelling data over time for blank TPU devices. Swelling was calculated according to the volume change. The error bars represent the standard deviation between the replicates (n=2). Only the device length was measured for this investigation, assuming all sides would swell equally. Volume calculations were done according to the length, width, and height increase.

With the hydrolysis prerequisite within the TPU drug delivery system via RAFT polymerization, a near-zero order release from HS-TPU was achieved, where RAL molecules without any modifications resulted in a burst effect.

### TPU Matrix Hydrophilicity Impact on Release Kinetics

Besides the hydrolysis requirement, MW was also helpful in the system since the RAL prodrug with an acetal linker followed a similar release trend as RAL within HS-TPU instead of p(RALSMA)13k (FIG. 7).

FIG. 7 is the cumulative RAL-ISO release from RAL-ISO embedded in HS-TPU in PBS media. There was no RAL release during the release period.

There has been a recent trend with generating prodrugs of water-soluble drugs to increase their retention within the body by increasing their hydrophobicity, where RAL has also been a focus point. Incorporating a prodrug within our dual-composite system compared to RAFT polymers to exhibit the usefulness of MW independent of the hydrolysis prerequisite was also investigated. RAL-ISO prodrug from Creighton et al. (2019) was chosen for this investigation because of its similar chemistry to a recent RAL prodrug created by Merck for increased retention time in the intestines and had a half-life of approximately 500 hours. RAL-ISO in HS-TPU displayed burst release like RAL, where nearly all prodrug molecules diffused from the matrix into the release media within the first two days (FIG. 7). It was anticipated that the prodrug's release profile to be more like RAL than p(RALSMA)13k despite the presence of the hydrolytic linker due to RAL-ISO and RAL molecules' very similar MW, 560.2 g/mol and 444.4 g/mol, respectively. Despite being slightly more hydrophobic than RAL, the prodrug still acted like a small water-soluble drug in our dual-composite delivery system.

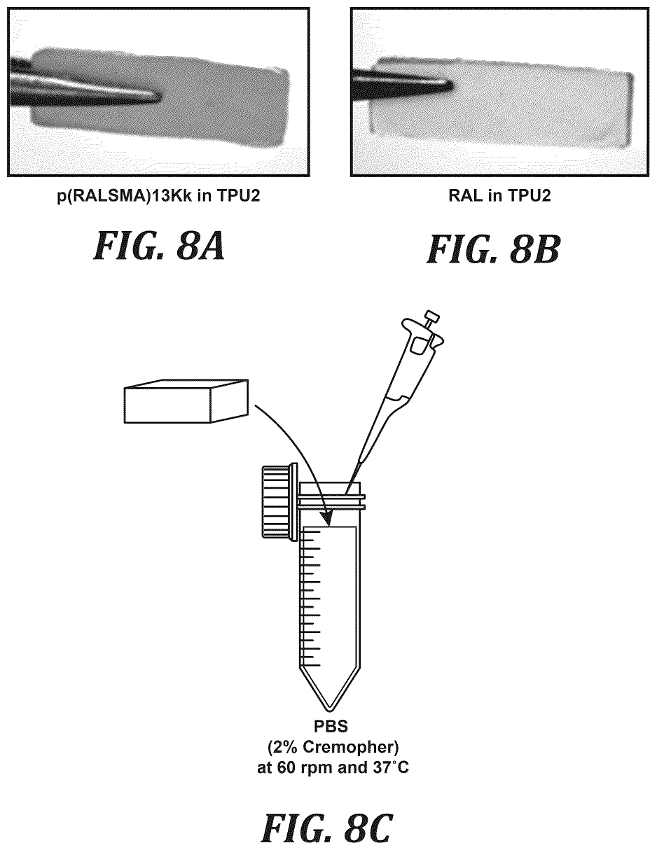

FIGS. 8A-8C shows example drug delivery systems including p(RALSMA)13k and RAL in HS-TPU, respectively.

### TPU Matrix Hydrophilicity Impact on Release Kinetics

Because of the usefulness of linker hydrolysis for RAL release within our dual-composite delivery system, different TPUs' water content and their impact the RAL release profile was investigated. Water saturation was a factor for RAL release and the remaining backbone behavior. TPUs have distinct chemical compositions that impact their water content following equilibrium. Two physiochemical features are hydrophilicity and crystallinity. Hydrophilicity mostly depends on the chosen polyol chemistry and MW, regarded as the “soft” segment, whereas crystallinity is often determined by the length and interspersion of “hard” isocyanate segments with the chain extenders. This investigation focused only on the hydrophilicity variable due to its direct correlation with TPU water content and, consequently, the mass change. Here, HS-TPU exhibited higher mass change compared to low swelling-(LS-)TPU and mid-swelling-(MS-)TPU. This was shown by measuring the water content within the TPU following equilibrium through how many water molecules were present in the devices (Table 1).

TABLE 1
TPU matrix hydrophilicity impact on release kinetics

| TPU Type | Lubrizol Characteristics | Mass Change (%) | |
|---|---|---|---|
| HS-TPU | Hydrophilic & High-Water | 104.11 | (+/−1.69) |
| | Uptake | | |
| LS-TPU | Hydrophobic | 1.66 | (+/−0.35) |
| MS-TPU | Hydrophilic & Low-Water | 42.6 | (+/−5.68) |
| | Uptake | | |

The more hydrophilic HS-TPU nearly doubled in its mass, but the more hydrophobic LS-TPU exhibited a nominal change. MS-TPU had a more prominent mass change than LS-TPU but was still smaller than HS-TPU, being between the two extremes (Table 1).

By investigating a TPU's mass change, we can make assumptions regarding its degree of hydrophilicity and how it might impact release within a system dependent on linker hydrolysis. For example, it was hypothesized that minimal mass change would result in no drug dissolution from the dual-composite system due to the insufficient water content for hydrolysis required for RAL release. The opposite was anticipated for TPUs with significant mass change, containing sufficient water content.

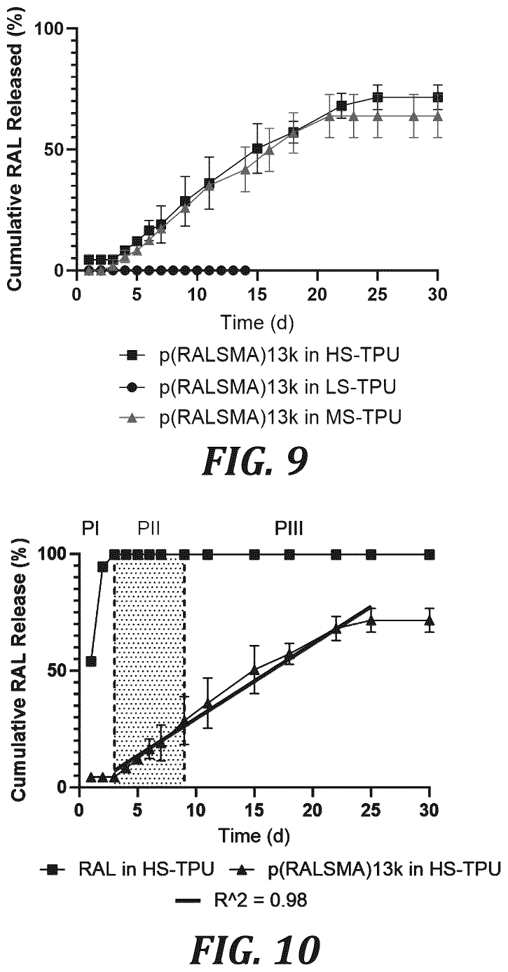

FIG. 9 is a graph showing RAL concentrations in PBS (pH=7.4) throughout the release study for RAL and p(RALSMA)13k in MS-TPU, HS-TPU, and LS-TPU at 37° C.

As mentioned, near zero-order RAL release was achieved in HS-TPU, which was also reflected with MS-TPU but not LS-TPU devices. LS-TPU devices embedded with p(RALSMA)13k showed no RAL release over the two-week study (FIG. 9). The lack of RAL release was expected due to the nominal mass change. Notably, the LS-TPU release profile suggested that p(RALSMA)13k was suspended in the matrix devices, forcing hydrolysis within the devices as proposed in Scheme 1 instead of outside the matrix device due to the high MW. MS-TPU's release profile was similar to HS-TPU's, exhibiting near-zero-order release with their kinetic time points. A primary difference between MS-TPU and HS-TPU release profiles was that MS-TPU drug dissolution reached its plateau earlier than HS-TPU at a lower cumulative RAL release percent (FIG. 9). These observations showed that the dual-composite delivery system depends on water content, where TPU hydrophilicity was helpful in determining the release profile, exhibiting a binary outcome. After reaching a certain water content threshold, different TPUs exhibit similar dissolution profiles with near-zero-order release. With this investigation, TPU hydrophilicity was determined to be another lever in determining the release profile that can be manipulated in combination with RAFT chemistry to tune drug release kinetics from our dual-composite delivery system.

FIG. 10 is a graph showing the cumulative release of RAL and p(RALSMA)13k over time in days.

Also, as shown earlier, there is a lag period before RAL release starts with the dual-composite system since full saturation allows for hydrolysis. This is also because of the dispersion of the p(RALSMA)13k throughout the devices. This example was only 1% weight loaded. The three-day lag period corresponds with the swelling data, where it took at least two days to reach full saturation.

The hydrolysis reaction was able to control the release achieving near-zero-order release. The lack of RAL release was expected due to the nominal mass change, where there is no water accumulation within the device and, as a result, no hydrolysis occurring.

Another observation from the LS-TPU investigation further supported that the p(RALSMA)13k was suspended within the matrix device due to its high MW, forcing hydrolysis to occur within the devices as depicted in the scheme earlier.

### Verification and Investigation of Polymeric Backbone Release Kinetics

The TPU water content was also analyzed in regard to the hydrolyzed p(RALSMA)13k backbone release from the matrix devices. Based on Phase III of Scheme 1, it was anticipated that there would be polymeric backbone release following RAL release due to the remaining negative charge on the backbone and its notably smaller MW around 4.5k g/mol (herein referred to as p(RALSMA)4.5k) compared to unhydrolyzed p(RALSMA)13k (Scheme S1). Following ester hydrolysis to release RAL, the remaining carboxylate groups on the polymeric backbone caused the polymer to gain a net negative charge, increasing its hydrophilicity as p(RALSMA)4.5k had a Log P of −0.63 while p(RALSMA)13k had a Log P larger than 6, being insoluble in water. Additionally, p(RALSMA)13k MW decreased considerably following RAL release because 65% of its total mass coming from attached RAL molecules. Thus, it was anticipated that p(RALSMA)4.5k would eventually diffuse out of the matrix devices.

For polymeric backbone investigation, RhMA fluorescence was measured as a proxy as it was bound to the backbone via a non-cleavable covalent bond. Emission was observed on Day 9 and every following timepoint with p(RALSMA)13k in HS-TPU media samples.

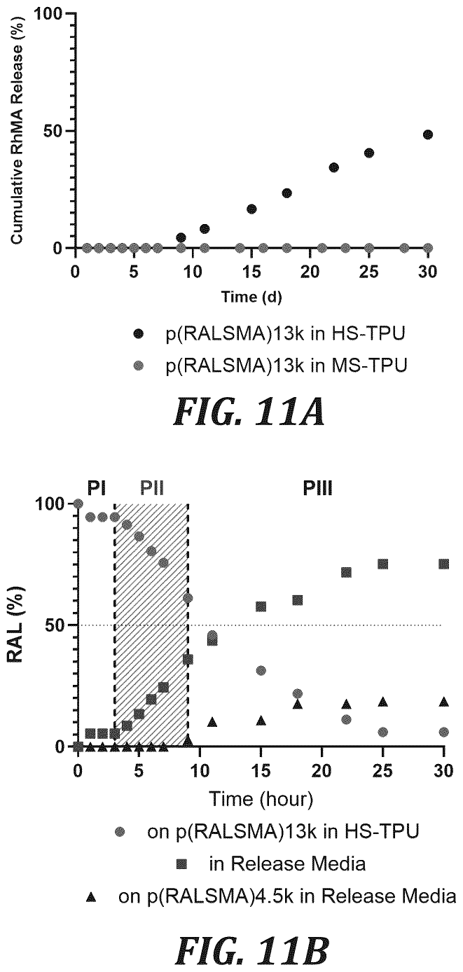

FIGS. 11A-11B are graphs of the location of p(RALSMA)13k, p(RALSMA)4.5k, and RAL throughout the release study. (A) RhMA concentration in PBS (pH=7.4) throughout the study for p(RALSMA)13k in HS-TPU and MS-TPU. (B) The remaining RAL percent and where it was located for one HS-TPU replicate throughout the study was calculated according to the RAL and RhMA concentrations in the release media and initial percent loading.

However, interestingly, there was no quantifiable emission from any samples from p(RALSMA)13k in MS-TPU throughout the study (FIG. 11A). The lag between the RAL and backbone release observed in the HS-TPU investigation supported the suggested mechanism where the unhydrolyzed p(RALSMA)13k were suspended within the matrix devices due to their high MW and hydrophobicity until reaching a certain RAL release threshold. The samples with RhMA emission were driven to 100% hydrolysis by incubation with a strong nucleophile to determine the suggested threshold. Further calculations with the p(RALSMA)13k, initial RAL in media, and following incubation RAL concentrations concluded that p(RALSMA)4.5k contained at most one unhydrolyzed prodrug linker after its dissolution from the matrix devices (Equations 1-3).

$$7 μg RhMA * 1 g / 1 E 6 μg * 1 mol RhMA / 666.2 g * 1 mol polymer / 0.03 mol RhMA * 1 mol monomer / 1 mol polymer * 20 mol RAL / 1 mol monomer = 7 E - 6 moles of RAL$$

$$7 μg RhMA * \frac{1 g}{1 E 6 μg} * \frac{1 mol RhMA}{666.2 g} * \frac{1 mol polymer}{0.03 mol RhMA} * \frac{1 mol monomer}{1 mol polymer} * \frac{20 mol RAL}{1 mol monomer} = 7 E - 6 moles of RAL \qquad Equation 1$$

$$19.77 μg RAL * \frac{1 g}{1 E 6 μg} * \frac{1 mol RAL}{444.4 g} = 4.45 E - 8 mol of RAL actually present \qquad Equation 2$$

$$( \frac{4.45 E - 8}{7 E - 6} ) * 1 \; 00 = 0.635 \% of RAL was present on the released backbones \qquad Equation 3$$

Equations 1-3 are calculations for the RAL cleavage threshold. Equation 1 depicts how much RAL should have been released if the released backbone was unhydrolyzed within the sample from Day 9 for replicate 2. Equation 2 depicts how much RAL was released following driving 100% hydrolysis with the sample from Day 9. Equation 3 depicts the percent of RAL that was actually present on the released backbone for the sample from Day 9 for replicate 2.

One replicate showed that at the end of the investigation, nearly 20% of the total RAL within the dual-composite delivery system was located on the released p(RALSMA)4.5k (FIG. 11B). It is significant to note that the RAL loading of each device was only one weight percent, where the TPU/p(RALSMA)13k ratio was large, potentially affecting the calculated helpful RAL cleavage threshold. With more space to diffuse through, more time will be spent within the fully saturated matrix device, where p(RALSMA)13k will continue to hydrolyze. There is a possibility that with a higher percent loading, the helpful RAL cleavage threshold could be smaller as there will be more p(RALSMA)13k towards the surface.

In contrast, the p(RALSMA)13k in MS-TPU release study refuted the assumption about the backbone release, as no quantifiable amount was observed in any release media sample (FIG. 11A). The only difference between the two studies is the type of TPU used in the matrix fabrication with their mass change following water equilibrium (Table 1). HS-TPU devices' masses doubled after reaching equilibrium, while MS-TPU devices increased by 0.5×. This observation suggested that the water content within the devices was also helpful in determining the p(RALSMA)4.5k release profile besides the hydrolyzed backbone's hydrophilicity and MW. Water molecules were relevant not only for hydrolysis but also for their hydrogen bonding capacity. It is well established in the literature and previous research papers that hydrogen bonding is highly significant in microphase separation in TPU systems. Hard segments, urethanes, within the TPU can make hydrogen bonds either between other urethanes or the soft segments, depending on the polyol chemistry. MS-TPU p(RALSMA)4.5k may have made hydrogen bonds with urethanes via its accessible carboxylates following ester hydrolysis, causing them to remain in the matrix device due to the newly formed hydrogen bond network. Because of the high water content within the HS-TPU devices, the proposed phenomenon may not occur in HS-TPU devices as the abundant water molecules would have made hydrogen bonds with carboxylates before the urethanes, possibly aiding in p(RALSMA)4.5k release from the matrix.

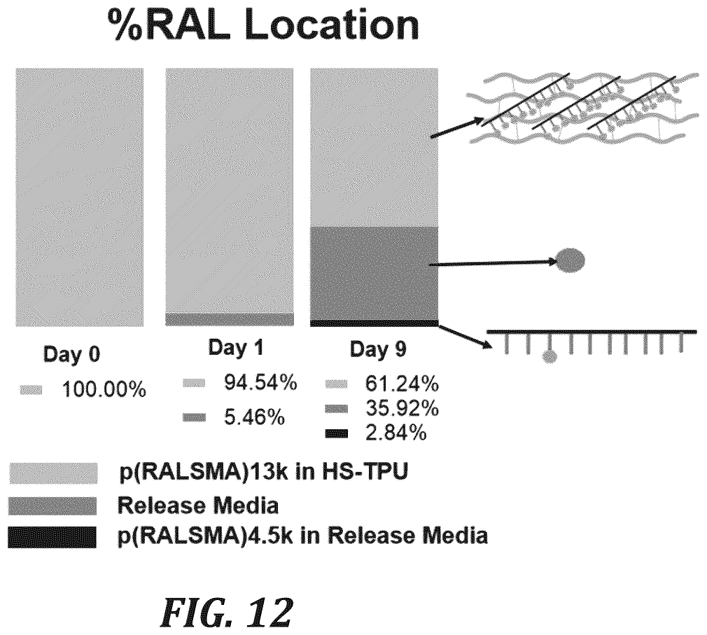

FIG. 12 is a graph showing the location of RAL each day.

Since polymeric backbone release was expected to occur following a certain RAL release threshold, the maximum RAL cleavage for this system was investigated. After the initial RAL release and fluorescence were measured, glycine was added to the collected samples as previously described. The hydrolysis data showed that not all released polymeric backbones are 100% cleaved.

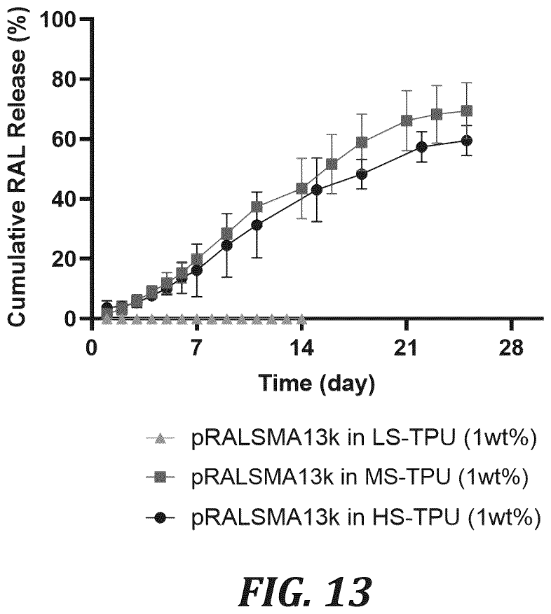

FIG. 13 is a graph showing the cumulative RAL release over time in multiple polymer matrices, in accordance with the present technology. As shown, water uptake and presence within the TPU matrix system dictated drug dissolution within the dual-composite delivery system, as hydrolysis was the rate-limiting step for release. Multiple types of TPUs were tested, as shown in Table 1.

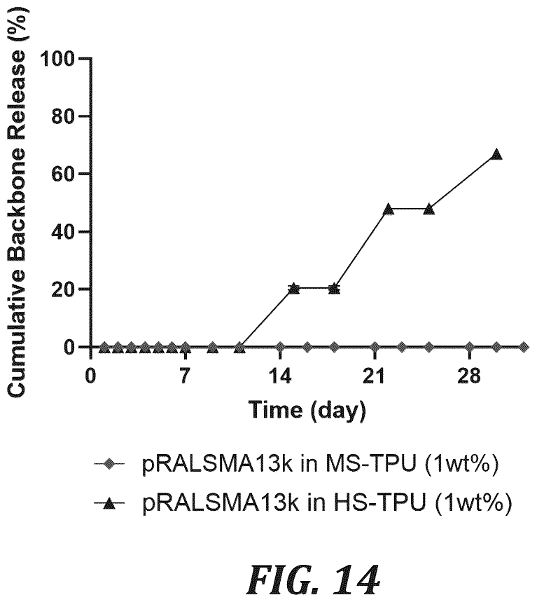

FIG. 14 is a graph showing the cumulative backbone release over time in multiple polymer matrices, in accordance with the present technology. The backbone release was also dependent on the matrix water uptake.

After the successful fabrication and results from the initial Dual-Composite Delivery System, the components of the system that impact the release behavior of the respective drug were investigated. It was determined that multiple components of the dual delivery system could be modified to achieve various results. For example, the drug used in the dual delivery system could be modified. Drugs with a modifiable functional group can be synthesized into a prodrug, regardless of hydrophilicity. For example, in some embodiments, the drug is RAL. In some embodiments, the drug is CMP.

Further, the linker chemistry can be altered to have different stimuli, such as water, reactive oxygen species (ROS), enzymes and the like. In some embodiments, the linkers may be selected from ester linkers or acetal carbonate linkers. In some embodiments, the linkers may include Self-immolative Spacers (SMA), Alanine-Cysteine-Methionine, and/or Acid-Cleavable-Moieties (ACM).

In some embodiments, the polymeric backbone may include Butyl Methacrylate (BMA), a homopolymer, Methyl Starch-graft Ethyl Methacrylate (MSEMA), or the like.

The polymer bulk chemistry and molecular weight can also be altered via RAFT for fabrication or therapeutic needs. Further, the matrix system itself can be altered to incorporate large molecules.

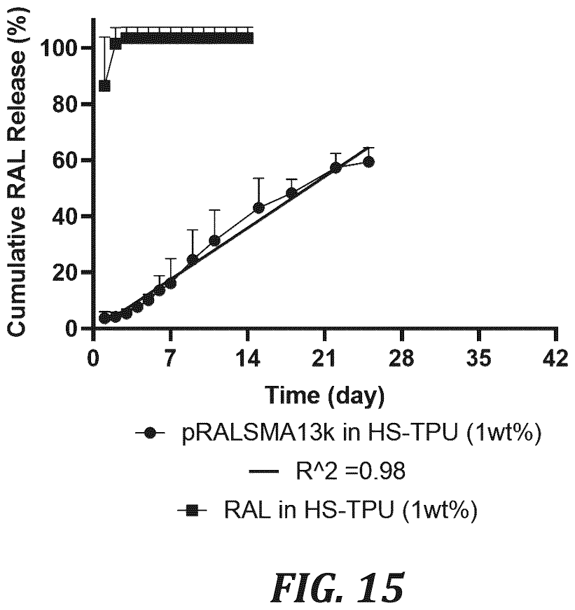

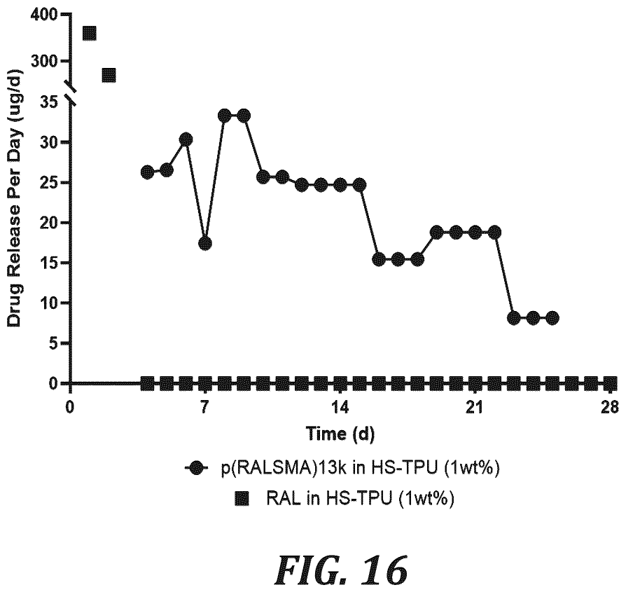

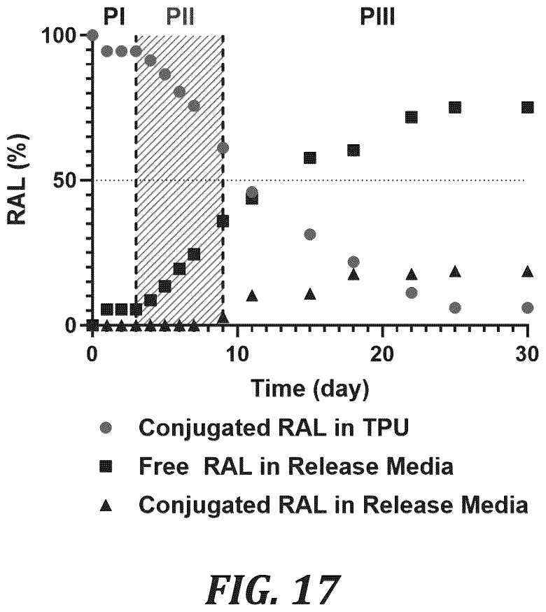

FIG. 15 is a graph showing the cumulative RAL release over time in multiple polymer matrices, in accordance with the present technology. FIG. 16 is a graph showing drug release per day, in accordance with the present technology. FIGS. 15-16 demonstrate the feasibility of controlling the release of RAL over time with the drug delivery system disclosed herein. FIG. 17 is a graph showing RAL release over time, in accordance with the present technology. As shown in FIG. 17, RAL release could be controlled by using the polymer matrix (TPU) as disclosed herein.

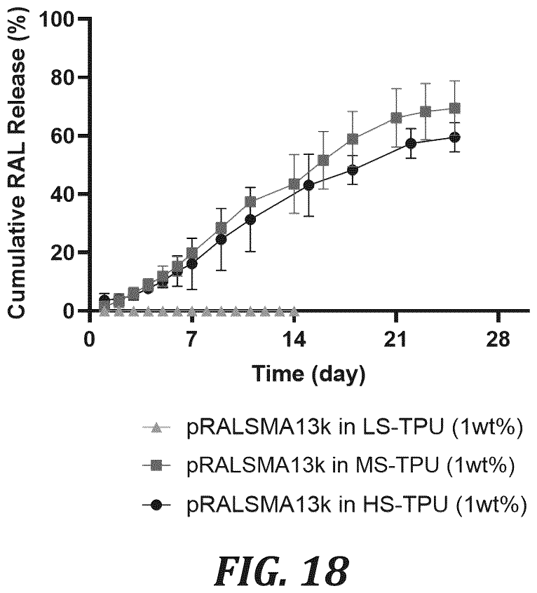

FIG. 18 is a graph showing RAL release over time, in accordance with the present technology. As shown in FIG. 18, altering the physicochemical characteristics of the drug delivery system is insufficient to achieve sustained release in the system (RALISO vs pRALSMA13k) (RALISO Log P=1.8). The system exhibits zero-order release when the molecular weight is increased via RAFT polymerization. The RAFT-drug polymer molecular exhibits a threshold where there is minimal difference between the three different drug-polymer release behaviors.

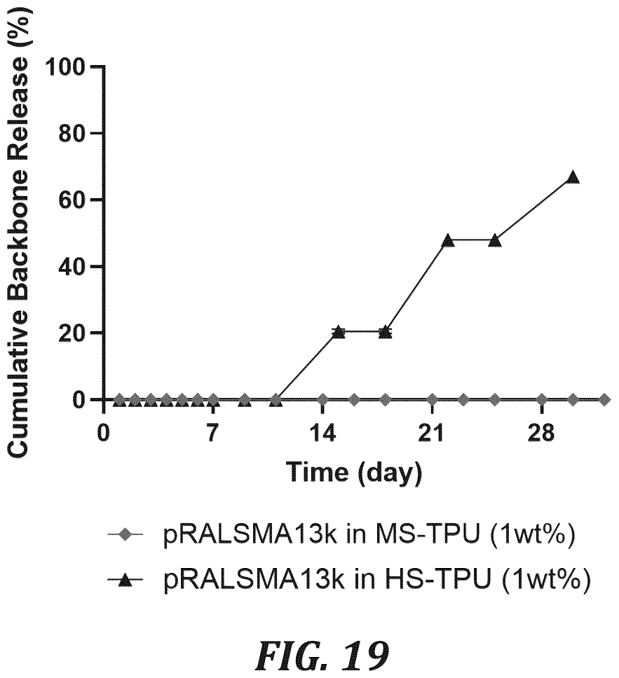

FIG. 19 is a graph showing the cumulative backbone release over time in multiple polymer matrices, in accordance with the present technology. The RAFT-drug polymer molecular weight affected the polymeric backbone release onset: pRALSMA23k showed no backbone release, whereas pRALSMA7k showed an earlier onset.

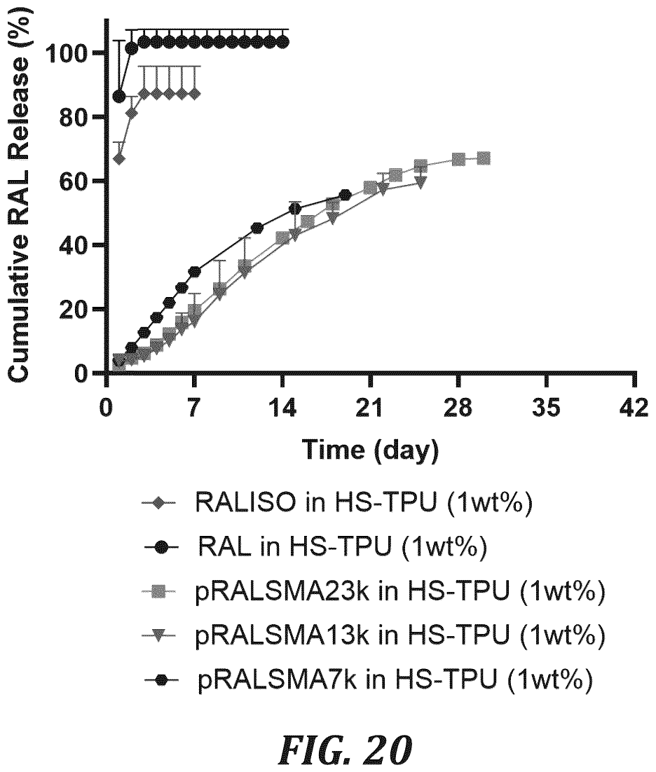

FIG. 20 is a graph showing RAL release over time, in accordance with the present technology. The RAFT-drug polymer chemistry impacts the drug release due to changes in net hydrophobicity within the TPU matrix system, where the more hydrophilic pRALSMA-MSEMA shows faster drug dissolution. Further, there is considerable backbone release from the MSEMA RAFT-drug polymer from day one.

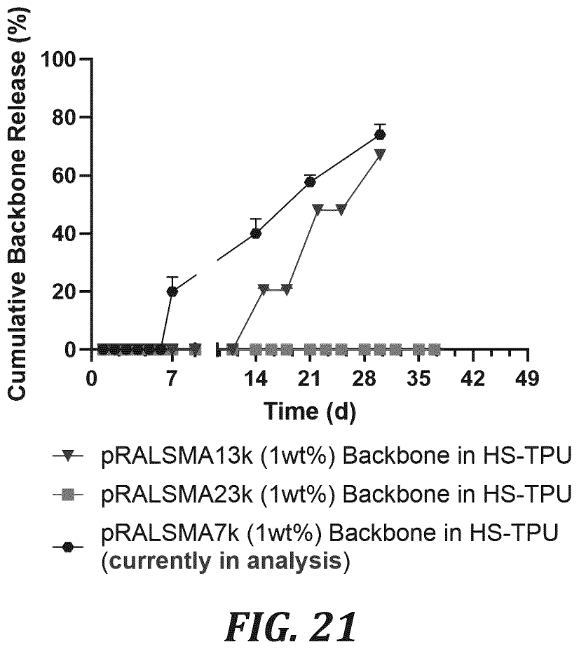

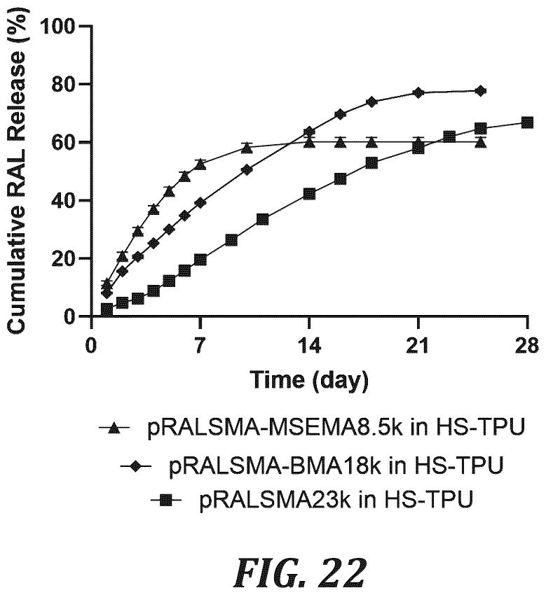

FIG. 21 is a graph showing RAL release over time, in accordance with the present technology. FIG. 22 is a graph showing RAL release over time, in accordance with the present technology.

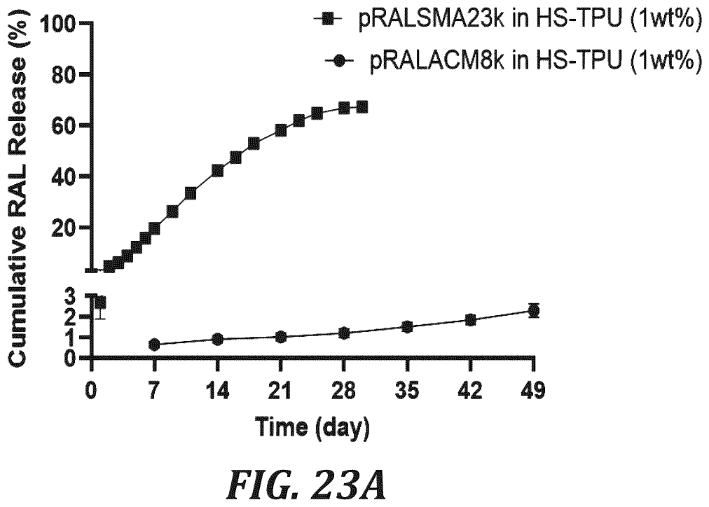

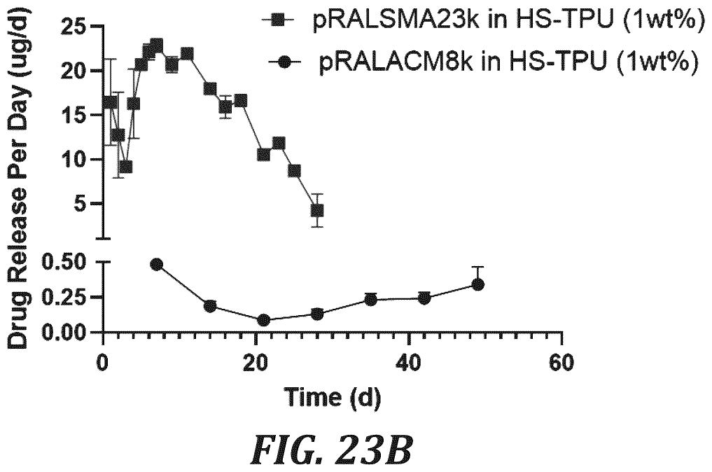

FIGS. 23A-23B are graphs showing cumulative RAL release over time, in accordance with the present technology. Linker chemistry allows for manipulating the release profile, where the acetal carbonate linker notably decreased the hydrolysis rate, and thereby, slowed the drug dissolution.

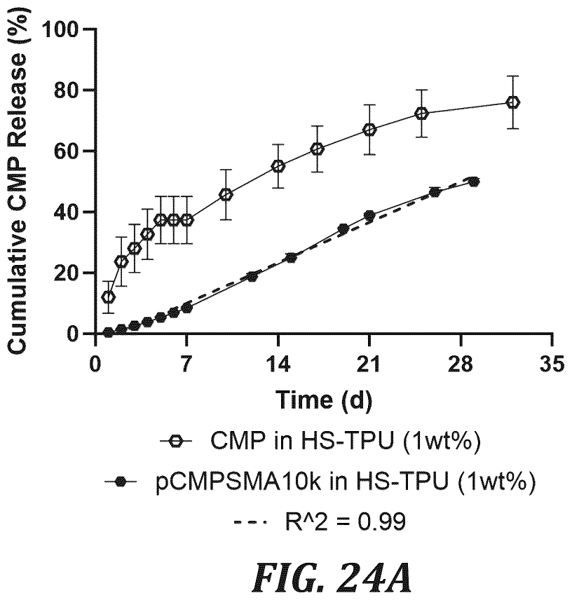

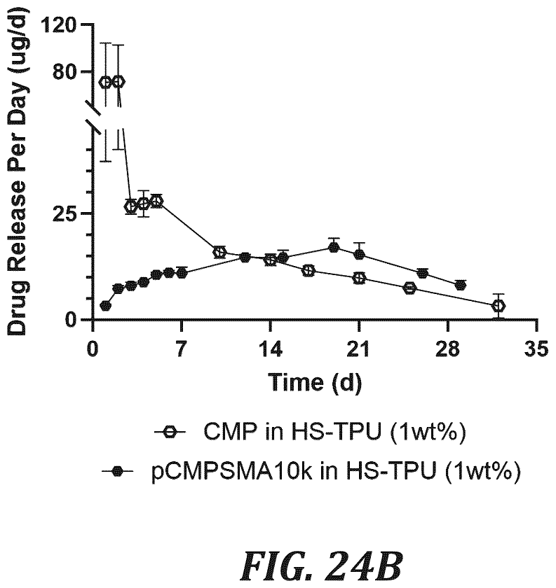

FIGS. 24A-24B are graphs showing cumulative CMP release over time, in accordance with the present technology. The drug delivery system was compatible with another small drug, 10-hydroxycampthotecin (CMP). The incorporation into the system resulted in a better sustained dissolution with zero-order release.

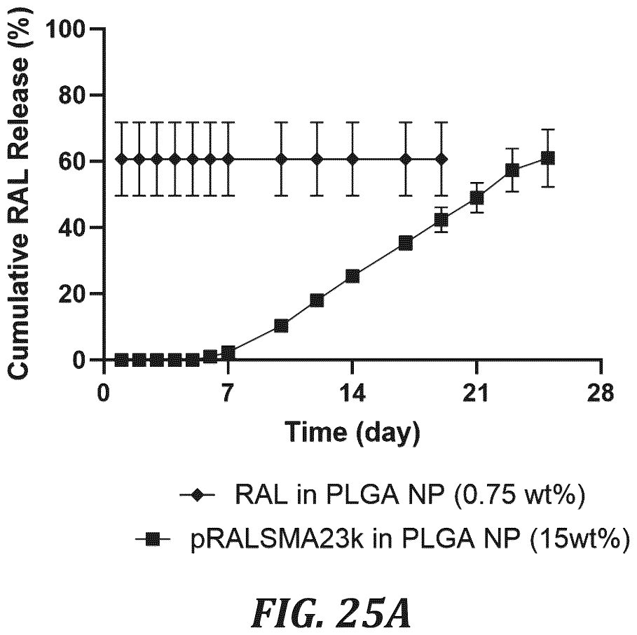

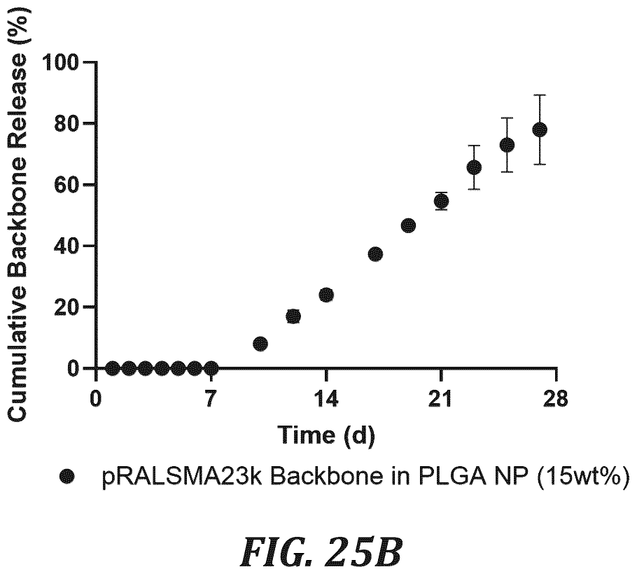

FIG. 25A-25B are graphs showing cumulative RAL release and cumulative backbone release over time, in accordance with the present technology. In some embodiments, the drug delivery system may be a PLGA injectable. The system, as a PLGA injectable, prevented burst release and provided sustained release.

While illustrative embodiments have been illustrated and described, it will be appreciated that various changes can be made therein without departing from the spirit and scope of the disclosure.

The present application may reference quantities and numbers. Unless specifically stated, such quantities and numbers are not to be considered restrictive, but representative of the possible quantities or numbers associated with the present application. Also, in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrase “at least one of A, B, and C,” for example, means (A), (B), (C), (A and B), (A and C), (B and C), or (A, B, and C), including all further possible permutations when greater than three elements are listed.

The detailed description set forth above in connection with the appended drawings, where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result. Generally, the embodiments disclosed herein are non-limiting, and the inventors contemplate that other embodiments within the scope of this disclosure may include structures and functionalities from more than one specific embodiment shown in the figures and described in the specification.

In the foregoing description, specific details are set forth to provide a thorough understanding of exemplary embodiments of the present disclosure. It will be apparent to one skilled in the art, however, that the embodiments disclosed herein may be practiced without embodying all the specific details. In some instances, well-known process steps have not been described in detail in order not to unnecessarily obscure various aspects of the present disclosure. Further, it will be appreciated that embodiments of the present disclosure may employ any combination of features described herein.

The present application may include references to directions, such as “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The present application may also reference quantities and numbers. Unless specifically stated, such quantities and numbers are not to be considered restrictive, but exemplary of the possible quantities or numbers associated with the present application. Also, in this regard, the present application may use the term “plurality” to reference a quantity or number. In this regard, the term “plurality” is meant to be any number that is more than one, for example, two, three, four, five, etc. The term “about,” “approximately,” etc., means plus or minus 5% of the stated value. The term “based upon” means “based at least partially upon.”

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

While illustrative embodiments have been illustrated and described, it will be appreciated that various changes can be made therein without departing from the spirit and scope of the disclosure.

What is claimed is:

1. A drug delivery system, comprising:
a swelling polymer matrix; and
a polymerized hydrophilic drug, wherein the drug delivery system is configured to control the release of the hydrophilic drug.

2. The drug delivery system of claim 1, wherein the drug delivery system is configured to achieve a near zero-order release of the hydrophilic drug.

3. The drug delivery system of claim 1, wherein the drug delivery system is configured to achieve a zero-order release of the hydrophilic drug.

4. The drug delivery system of claim 3, wherein the drug delivery system is hydrophilic drug has an R² value greater than 0.949.

5. The drug delivery system of claim 1, wherein the hydrophilic drug is polymerized with reversible addition fragmentation chain transfer (RAFT) polymerization.

6. The drug delivery system of claim 1, wherein controlling the release of the hydrophilic drug comprises:
reaching a water equilibrium within the swelling polymer matrix;
hydrolyzing the polymerized hydrophilic drug within the swelling polymer matrix; and
diffusing the hydrophilic drug from the swelling polymer matrix.

7. The drug delivery system of claim 1, wherein the hydrophilic drug has a molecular weight of about 100-1000 g/mol (0.1-1 kDa).

8. The drug delivery system of claim 1, wherein the hydrophilic drug is raltegravir (RAL).

9. The drug delivery system of claim 1, wherein the hydrophilic drug is 10-hydroxycampthotecin (CMP).

10. The drug delivery system of claim 1, wherein the polymerized hydrophilic drug comprises comprising a polymeric backbone with hydrolytic linkers.

11. The drug delivery system of claim 10, wherein the hydrolytic linkers are ester linkers.

12. The drug delivery system of claim 10, wherein the hydrolytic linkers are acetal carbonate linkers.

13. The drug delivery system of claim 1, wherein the swelling polymer matrix is a Poly(lactic-co-glycolic acid) (PLGA) matrix.

14. The drug delivery system of claim 1, wherein the swelling polymer matrix is a thermoplastic polyurethane (TPU) matrix.

15. The drug delivery system of claim 14, wherein the TPU matrix is a high-swelling (HS) TPU matrix.

16. The drug delivery system of claim 14, wherein the TPU matrix is selected from PT42DE35 and PY-PT83AE35.

17. The drug delivery system of claim 1, wherein the drug delivery system is configured to release the hydrophilic drug over a duration of about 30 days.

18. A method of using the drug delivery system of claim 1, the method comprising:
providing the polymerized hydrophilic drug dispersed within the swelling polymer matrix;
reaching a water equilibrium within the swelling polymer matrix;
hydrolyzing the polymerized hydrophilic drug; and
diffusing the hydrophilic drug outside of the swelling polymer matrix.

19. The method of claim 18, wherein the hydrophilic drug is released at an effective level for about 30 days.

20. A method of making the drug delivery system of claim 1, the method comprising:
polymerizing a hydrophilic drug to produce a polymerized hydrophilic drug; and
embedding the polymerized hydrophilic drug into a swelling polymer matrix.
